(19) United States
(12) Patent Application Publication
(10) Pub. No.: US 2024/0156635 A1
(43) Pub. Date: May 16, 2024

(54) METHODS AND DYNAMIC DENTAL APPLIANCE FOR THE TREATMENT OF AWAKE BRUXISM AND/OR SLEEP BRUXISM

(71) Applicant: Population Sleep, LLC, Dallas, TX (US)

(72) Inventor: Rolf MAIJER, Victoria (CA)

(21) Appl. No.: 18/510,703

(22) Filed: Nov. 16, 2023

Related U.S. Application Data
(60) Provisional application No. 63/425,941, filed on Nov. 16, 2022.

(51) Int. Cl. A61F 5/56

(52) U.S. Cl. CPC A61F 5/566; A61F 2005/563

(57) ABSTRACT

A dynamic dental appliance for bruxism treatment comprises a base configured to fit on either the upper dental arch or the lower dental arch of an individual; a pair of occlusal pads, one of the occlusal pads configured to overlie the left posterior teeth and the other of the occlusal pads configured to overlie the right posterior teeth; and a biasing mechanism acting between the base and the occlusal pads, the biasing mechanism configured to exert an adjustable force against the occlusal pads when the occlusal pads are moved from a flat orientation towards a generally angled activated position.

### CROSS-REFERENCE TO RELATED APPLICATION

This application claims the benefit of U.S. Provisional Application No. 63/425,941 filed on Nov. 16, 2022, the entire content of which is incorporated herein by reference.

### FIELD

The subject disclosure relates to methods and dynamic dental appliance for the treatment of awake bruxism and/or sleep bruxism.

### BACKGROUND

Bruxism is a condition defined as a repetitive jaw-muscle activity characterized by clenching or grinding of the teeth and/or by bracing or thrusting of the mandible. This repetitive jaw-muscle activity may occur when individuals are awake (awake bruxism) or during sleep (sleep bruxism). Whereas individuals may be aware of bruxing activity when awake, they may not be aware of this involuntary activity when it occurs during sleep. Bruxism can result in the chronic overloading of the stomatognathic system, including the teeth, jaw joints, and associated musculoskeletal structures. Over time, heavy intermittent forces exerted during bruxism pose a risk to teeth and can produce one or more negative effects on the oral environment. For example, bruxism can cause excessive tooth wear, periodontal recession, broken dental fillings, and fractured or cracked teeth, all which represent a distinct threat to oral health. In situations where dental implants are placed in the bony structures, bruxism may loosen or dislodge expensive dental prostheses including the surgically placed anchors, the abutments that connect the implants to a crown or bridge, and/or the crowns or bridges due to the heavy excessive forces placed on them. The excessive jaw-muscle activity and excessive loading of the stomatognathic system can also fatigue the muscles of mastication and can result in chronic pain in the masticatory structures (neck and facial muscles as well as the jaw joints). Chronic bruxism can contribute to changes in the jaw joint that affect normal jaw function, cause pain, or both. It can also trigger the onset of temporomandibular disorders (TMD) and aggravate preexisting TMDs.

Sleep bruxism is considered a sleep-related movement disorder that can negatively impact health and is characterized by teeth grinding and clenching during sleep. There is a neurological component of sleep bruxism that drives an involuntary, spontaneous reflex or movement of the jaw. Objective measurements have shown that occlusal (biting) forces can be much greater during sleep bruxism than those exerted during awake bruxism. Sleep bruxism is associated with temporomandibular pain, headaches, tooth wear, and disruption of the bed partner's sleep. Sleep bruxism most often occurs with or following unconscious sleep-related microarousals. In addition to the potential damage to orofacial structures, sleep bruxism impairs sleep architecture, resulting in adverse changes in sleep parameters such as decreased total sleep time, decreased sleep efficiency, and greater sleep fragmentation. Sleep-related microarousals related to sleep bruxism are a root cause of sleep fragmentation and non-restorative and non-refreshing sleep. Sleep bruxism is associated with other sleep disorders including insomnia, especially in middle-aged females, and people with obstructive sleep apnea. Over time, chronic sleep bruxism compromises both physical and mental health as well as daytime function. Compared to those without sleep bruxism, individuals with sleep bruxism experience poorer perceived sleep quality; higher levels of daytime sleepiness; greater fatigue intensity; and more affective symptoms such as depression, anxiety, and irritability. Chronic sleep fragmentation has a cumulative effect resulting in chronic partial sleep deprivation, cognitive impairment, and decreased quality of life.

There are a number of treatment modalities for the treatment of awake and sleep bruxism and the treatment modality selected for an individual is often dependent on the health professional that has been consulted for treatment. These interventions can be grouped into the following categories: Dental Devices, Medical Devices, Biofeedback Devices, Psychological Interventions, and Pharmacological Interventions.

Dental devices used to treat bruxism embody devices or material worn between the teeth during the day or night that protect the teeth from the deleterious effects of clenching and are a modality for preventing damage to dental structures and intraoral tissue. In many instances, the use of occlusal dental splints is the first line of attack. These occlusal dental splints are typically of a one-piece construction, are formed of plastic material, and are configured to fit on either the upper dental arch or the lower dental arch. Depending on the material used, the muscles may respond differently in frequency and force exerted against the occlusal dental splints. These dental orthotics are static in nature and consist of a solid structure with no moving parts.

Non-custom, prefabricated dental devices are available over-the-counter for the treatment of teeth grinding and bruxism. Dentists routinely offer patients treatment with a custom dental device to reduce harm on dental and intraoral structures, alleviate pain in the muscles of mastication, reduce headaches, etc. The most common appliance delivered by dentists is a custom-made, horseshoe shaped device that covers a full dental arch of the individual wearing the dental device. Occlusal dental devices like this may be worn only on the full upper dental arch, the full lower dental arch, or both the upper and lower dental arches concurrently. These occlusal dental devices may have features such as ramps or ridges that guide or control the movement of the jaw during bruxism. Dental devices that cover only the anterior teeth of the wearer also exist.

While occlusal dental devices worn during wake or sleep offer dental protection, there is insufficient evidence to show that these occlusal dental devices worn during sleep effectively reduce or eliminate sleep bruxism and related clinical sequelae such as sleep-bruxism related sleep arousals. There is also conflicting evidence that these occlusal dental devices improve jaw function, reduce pain, improve sleep quality, or alleviate daytime symptoms of non-restorative sleep.

Biofeedback devices used to treat bruxism encompass several types of devices that provide biofeedback in response to a bruxism event to stop or lessen the clenching force. One type of biofeedback device relies on the external measurement of movement in the
muscles of mastication to provide a biofeedback signal to the user. Another type of biofeedback device involves the integration of electronics into an intraoral appliance or occlusal guard.

Medical devices such as mandibular advancement devices (MAD) used for the treatment of snoring and obstructive sleep apnea have been shown to also reduce sleep bruxism. MADs reposition the jaw in a forward position and prevent the jaw from falling back. Some MADs allow the jaw the ability to move freely side to side while others limit the movement of the jaw in any direction. However, there can be negative side-effects of limiting jaw movement in any direction if sleep bruxism is present. The most commonly prescribed therapy for obstructive sleep apnea, Continuous Positive Airway Pressure (CPAP) therapy, has also been shown to reduce sleep bruxism. However, neither CPAP nor MAD devices address movement or clenching activity that occurs independently of an apneic or hypopneic event, nor any non-respiratory cause of sleep arousal.

Psychological interventions are also used to treat bruxism. If stress is considered the primary contributing factor, cognitive behavioral therapy (CBT) is usually recommended. CBT interventions have reported limited success rates as well.

Pharmacological interventions such as medications prescribed by health professionals are also used to treat bruxism. Some medication, such as benzodiazepines and muscle relaxants, are used to treat pain and reduce bruxism-related motor activity. However, pain medications can have deleterious side effects ranging from REM suppression and morning hypotension to addiction. Chronic pain in the jaw muscles is often treated with bimonthly Botox injections. Recent research has shown that Botox injections do not reduce the genesis of bruxism but simply dampen the intensity of the clench. Other medications target sleep rather than muscle activity to improve sleep initiation and continuity as well as the consequences of insufficient restorative sleep, such as daytime fatigue and brain fog.

As will be appreciated, improvements in the treatment of bruxism are desired. It is therefore an object to provide novel methods and dynamic dental appliance for the treatment of both awake and sleep bruxism.

This background serves only to set a scene to allow a person skilled in the art to better appreciate the following brief and detailed descriptions. Therefore, none of the above discussion should necessarily be taken as an acknowledgement that this discussion is part of the state of the art or is common general knowledge.

### BRIEF DESCRIPTION

It should be appreciated that this brief description is provided to introduce a selection of concepts in a simplified form that are further described below in the detailed description. This brief description is not intended to be used to limit the scope of claimed subject matter.

Accordingly, in one aspect there is provided a dynamic dental appliance for bruxism treatment comprising: a base configured to fit on either the upper dental arch or the lower dental arch of an individual; a pair of occlusal pads, one of the occlusal pads configured to overlie the left posterior teeth and the other of the occlusal pads configured to overlie the right posterior teeth; and a biasing mechanism acting between the base and the occlusal pads, the biasing mechanism configured to exert an adjustable force against the occlusal pads when the occlusal pads are moved from a flat orientation towards a generally angled activated position.

In one or more embodiments, the biasing mechanism comprises a pair of adjustable spring mechanisms, each adjustable biasing mechanism acting between the base and a respective one of the occlusal pads.

In one form, each spring mechanism comprises at least one spring acting between the base and the respective one of the occlusal pads. Each spring may be a coil spring.

In another form, each adjustable spring mechanism comprises a pair of springs acting between the base and the respective one of the occlusal pads, the springs being positioned on the respective buccal and lingual sides of the dynamic dental appliance. Each spring may be a coil spring.

In one form, the fulcrum of the spring mechanisms is located at the anterior end of each occlusal pad.

In one form, in the activated position, the angle of the occlusal pads is in the range of from about 5 degrees to about 45 degrees.

In one or more embodiments, the base is configured to fit passively on either the upper dental arch or the lower dental arch.

In one form, the base is in the form of a tray comprising an anterior portion configured to overlie the anterior teeth and posterior portions configured to run along the lingual sides of posterior teeth, the buccal sides of posterior teeth or both the lingual and buccal sides of posterior teeth.

In one or more embodiments, the occlusal pads are flat and smooth to allow the individual's teeth to skate freely thereacross.

In one or more embodiments, the exerted adjustable force is in the range of from about 0.1 Newton to about 400 Newtons.

According to another aspect there is provided a bruxism treatment method comprising: using at least one dynamic dental appliance as described above installed in an individual's mouth and fitted on either the upper dental arch, the lower dental arch or both the upper and lower dental arches that exerts an adjustable force between the individual's jaws during clenching to resist the clenching and counter brain-driven clench stimulus.

In one or more embodiments, the exerted adjustable force is in the range of from about 0.1 Newton to about 400 Newtons.

According to another aspect there is provided a bruxism treatment method comprising: using at least one dynamic dental appliance fitted on either the upper dental arch, lower dental arch or both the upper and lower dental arches of an individual that exerts an adjustable force between the individual's jaws during clenching to resist the clenching such that the dynamic dental appliance absorbs and dissipates the clenching force.

In one or more embodiments, the exerted adjustable force is in the range of from about 0.1 Newton to about 400 Newtons.

According to another aspect there is provided a bruxism treatment method comprising using at least one dynamic dental appliance fitted on a dental arch of an individual that exerts an adjustable force between the individual's jaws during clenching to resist the clenching and counter brain-drive clench stimulus during sleep.

In one or more embodiments, the exerted adjustable force is in the range of from about 0.1 Newton to about 400 Newtons.

In one or more embodiments, the method comprises fitting the dynamic dental appliance on either (i) the lower dental arch, (ii) the upper dental arch or (iii) both the upper and lower dental arches.

As will be appreciated by those of skill in the art, the subject dynamic dental appliance sends a natural biofeedback signal to the brain to reduce, lessen or stop the clenching or grinding of the teeth and/or bracing or thrusting of the mandible. The subject dynamic dental appliance also absorbs and dissipates clenching forces, which reduces and prevents overloading of the stomatognathic system thereby reducing or eliminating subsequent clinical sequalae such as damage to intraoral structures and chronic pain. During sleep, the reduction of bruxism activity has the added benefit of improving sleep quality and continuity. The subject dynamic dental appliance and methods of bruxism treatment allow awake and/or sleep bruxism to be treated using a non-pharmacological solution (e.g. an alternative to opioids, sleeping pills), a non-invasive therapy (e.g. an alternative to Botox injections), and a non-electronic intervention (to avoid safety concerns associated with electronics in an intraoral environment such as corrosion and electric shock).

### BRIEF DESCRIPTION OF THE DRAWINGS

Embodiments will now be described by way of example only with reference to the accompany drawings in which:

FIG. 1 is a top-side perspective view of a dynamic dental appliance in accordance with the subject disclosure;

FIG. 2 is a side elevational view of the dynamic dental appliance of FIG. 1, the opposite side elevational view being a mirror image;

FIG. 3 is a bottom view of the dynamic dental appliance of FIG. 1;

FIG. 4 is a side elevational, lateral skull view showing the dynamic dental appliance of FIG. 1 fitted on the lower dental arch of an individual and configured with its occlusal pads in an activated position, only one of which is shown; and

FIG. 5 is an opposite side elevational, lateral skull view showing the dynamic dental appliance of FIG. 1 fitted on the lower dental arch of an individual and configured with its occlusal pads in a non-activated, passive flat orientation, only one of which is shown.

### DETAILED DESCRIPTION

The foregoing brief description, as well as the following detailed description of certain examples will be better understood when read in conjunction with the appended drawings. As used herein, an element or feature introduced in the singular and preceded by the word “a” or “an” should be understood as not necessarily excluding the plural of the elements or features. Further, references to “one example” or “one embodiment” are not intended to be interpreted as excluding the existence of additional examples or embodiments that also incorporate the described elements or features. Reference herein to “example” means that one or more feature, structure, element, component, characteristic and/or operational step described in connection with the example is included in at least one embodiment and/or implementation of the subject matter according to the subject disclosure. Thus, the phrases “an example,” “another example,” and similar language throughout the subject disclosure may, but do not necessarily, refer to the same example. Further, the subject matter characterizing any one example may, but does not necessarily, include the subject matter characterizing any other example.

Unless explicitly stated to the contrary, examples or embodiments “comprising” or “having” or “including” an element or feature or a plurality of elements or features having a particular property may include additional elements or features not having that property. Also, it will be appreciated that the terms “comprises”, “has”, “includes” means “including but not limited to” and the terms “comprising”, “having” and “including” have equivalent meanings.

As used herein, the term “and/or” can include any and all combinations of one or more of the associated listed elements or features.

Reference herein to “configured” denotes an actual state of configuration that fundamentally ties the element or feature to the physical characteristics of the element or feature preceding the phrase “configured to.”

It will be understood that when an element or feature is referred to as being “on”, “attached” to, “connected” to, “coupled” to, “contacting”, “fixed” to etc. another element or feature, that element or feature can be directly on, attached to, connected to, coupled to, or contacting the other element or feature or intervening elements may also be present. In contrast, when an element or feature is referred to as being “directly” on, “directly attached” to, “directly connected” to, “directly coupled” to, “directly contacting”, “directly fixed” to etc. another element or feature, there are no intervening elements or features present. Similarly, it will be understood that when an element is referred to as being “directly between” other elements, that element is positioned between the other elements without any intervening elements. In contrast, when an element is referred to as being “between” other elements, that element is positioned between the other elements but intervening elements may also be present.

It will be understood that spatially relative terms such as “bottom”, “under”,
“below”, “lower”, “over”, “upper”, “top”, ““front”, “back”, “side” and the like, may be used herein for ease of describing the relationship of an element or feature to another element or feature as depicted in the figures. The spatially relative terms can however, encompass different orientations in use or operation in addition to the orientation depicted in the figures.

As used herein, the terms “approximately”, “about”, “generally”, “substantially” etc. represent an amount or characteristic close to the stated amount or characteristic that still performs the desired function or achieves the desired result. For example, the terms “approximately” and “about” in reference to a stated amount include amounts that are within engineering or design tolerances of the stated amount that would be readily appreciated by a person skilled in the art. Similarly, for example, the term “substantially” in reference to a stated characteristic of an element includes elements that nearly completely provide the stated characteristic, and the term “generally” in reference to a stated characteristic of an element includes elements that predominately provide the stated characteristic.

Unless otherwise indicated, the terms “first”, “second” etc. are used herein merely as labels, and are not intended to impose ordinal, positional, or hierarchical requirements on the elements to which these terms refer. Moreover, reference to a “second” element does not require or preclude the existence of a lower-numbered element (e.g., a “first” element) and/or a higher-numbered element (e.g., a “third” element).

In the subject disclosure, a dynamic dental appliance for awake and/or sleep bruxism treatment is described that comprises a base configured to fit on either the upper dental arch or the lower dental arch of an individual; a pair of occlusal pads, one of the occlusal pads configured to overlie the left posterior teeth and the other of the occlusal pads configured to overlie the right posterior teeth; and a biasing mechanism acting between the base and the occlusal pads, the biasing mechanism configured to exert an adjustable force against the occlusal pads when the occlusal pads are moved from a flat orientation towards a generally angled activated position. In order to treat bruxism, the dynamic dental appliance is fitted on either the upper dental arch or the lower dental arch or both dental arches of an individual. When bruxism occurs, the muscles of mastication contract and load the dentition. The dynamic dental appliance in an activated state absorbs and dissipates the force of the individual's jaws during clenching. This resistance to clenching creates a non-electronic biofeedback loop that transmits a signal to the individual's brain to interrupt the clenching, lessen the intensity of the clenching, and/or reduce the frequency of the clenching. Further specifics of the dynamic dental appliance and its use for awake and/or sleep bruxism treatment will now be described.

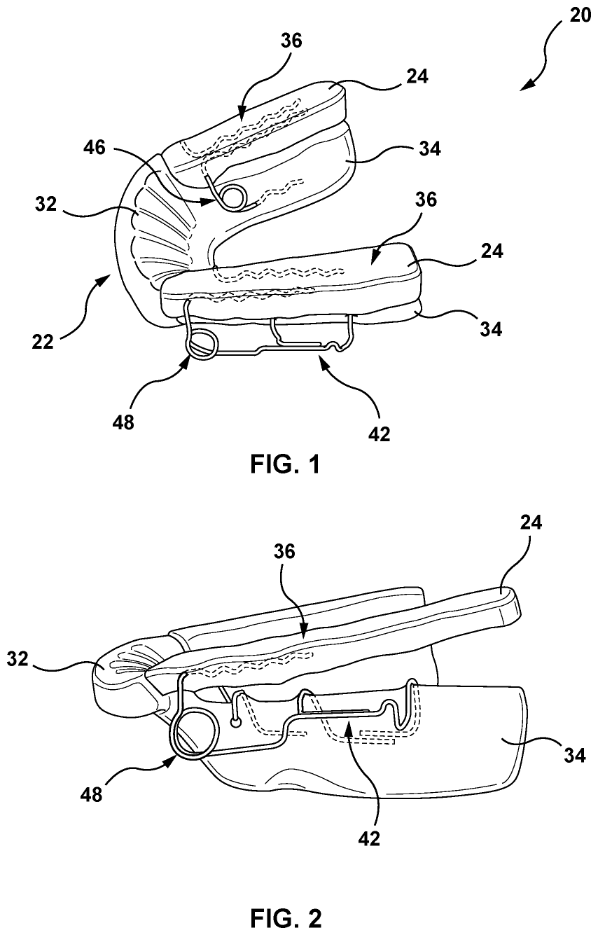

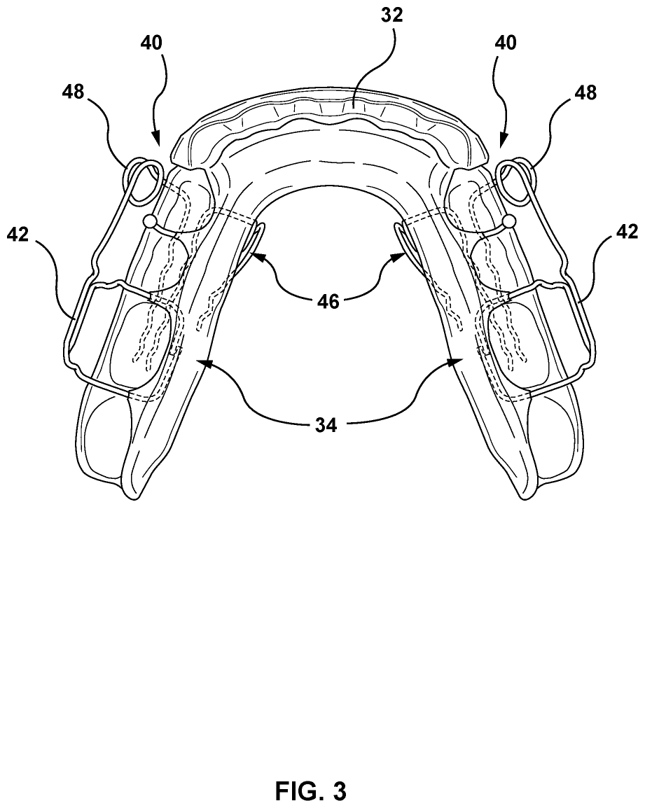

Turning now to FIGS. 1 to 3, an exemplary dynamic dental appliance for bruxism treatment is shown and is generally identified by reference numeral 20. The dynamic dental appliance 20 is configured to fit on either the upper teeth dental arch or the lower dental arch. For ease of description only, the dynamic dental appliance 20 will be described with reference to the figures as if intended to be fitted on the lower dental arch of an individual. It will however be appreciated by those of skill in the art that the dynamic dental appliance 20 can equally be fitted on the upper dental arch of the individual, in which case, described upward orientations will be the opposite to those described below (i.e., downward).

As can be seen, the dynamic dental appliance 20 comprises a base 22 in the form of a retention tray or frame, a pair of laterally spaced, distally extending occlusal pads 24, and a biasing mechanism acting between the base 22 and the occlusal pads 24.

In this embodiment, the base 22 is in the form of an arch-shaped or U-shaped, one-piece retention tray or frame formed of plastic material such as acrylic or other suitable material. The retention tray 22 is configured to fit passively but snugly on the lower dental arch of the individual. In particular, the retention tray 22 comprises an anterior portion 32 configured to cover the incisal and lingual surfaces of the anterior lower teeth and posterior portions 34 configured to cover the lingual surfaces of the posterior lower arch.

The occlusal pads 24 in this embodiment are formed of a resilient plastic material such as acrylic or other suitable material and are posteriorly angled. The occlusal pads 24 are separate from the retention tray 22 and are configured to overlie the occlusal surfaces of posterior lower teeth. One occlusal pad is configured to overlie the left posterior lower teeth and one occlusal pad is configured to overlie the right posterior teeth. Each occlusal pad 24 has a major generally planar, flat, and smooth upper surface 36. The upper surfaces 36 are configured to contact the occlusal surfaces of opposing posterior upper teeth as will be described. The flat and smooth major surfaces 36 of the occlusal pads 24 allow the individual's teeth to skate freely across the major surfaces 36. The major surfaces 36 of the occlusal pads 24 are devoid of any grooves that may impede movement of the individual's teeth or jaw and there are no slots, ramps, hooks, bars, or other features of the dynamic dental appliance 20 that act to reposition or retain the individual's jaw in a specific place or restrict the jaw's plane of movement side to side or forward and backward.

The biasing mechanism in this embodiment comprises a pair of spring mechanisms 40 with each spring mechanism 40 acting between the retention tray 22 and a respective one of the occlusal pads 24. In this embodiment, the spring mechanisms 40 are formed of stainless steel, NiTi, plastic, or other suitable material.

In this embodiment, each spring mechanism 40 comprises lingual and buccal coil springs 46 and 48, respectively. Each lingual and buccal coil spring 46 and 48 in this embodiment comprises a single turn coil although those of skill in the art will appreciate that the lingual and buccal coil springs may comprise additional turns. The lingual and buccal coil springs 46 and 48 are positioned adjacent the anterior portion 32 of the retention tray 22. As a result, the fulcrum of each spring mechanism 40 is located at the anterior end of each occlusal pad 24.

Each lingual coil spring 46 is fixedly secured adjacent one end to the retention tray 22 and adjacent the other end to a respective occlusal pad 24. In this embodiment, the ends of the lingual coil springs 46 are embedded into both the posterior portions 34 of the retention tray 22 and the occlusal pads 24 although those of skill in the art will appreciate that the lingual coil springs 46 may be affixed to the retention tray 22 and/or occlusal pads 24 using other suitable methods such as non-toxic adhesives, ultrasonic or friction lap welding etc. Each buccal coil spring 48 is fixedly secured adjacent one end to the respective occlusal pad 24. In this embodiment, the buccal coil springs 48 are embedded into the occlusal pads 24 although those of skill in the art will appreciate that the buccal coil springs 48 may be affixed to the occlusal pads 24 using other suitable methods such as non-toxic adhesives, ultrasonic or friction lap welding etc. The other ends of the buccal coil springs 48 are anchored to retainer clasps 42 in the form of Adams clasps that are embedded into the posterior portions 34 of the retention tray 22. In this embodiment, each buccal coil spring 48 is welded to a respective retainer clasp 42 although those of skill in the art will appreciate that the buccal coil springs 48 may be affixed to the retainer clasps 42 using other suitable methods such as non-toxic adhesives, ultrasonic or friction lap welding etc.

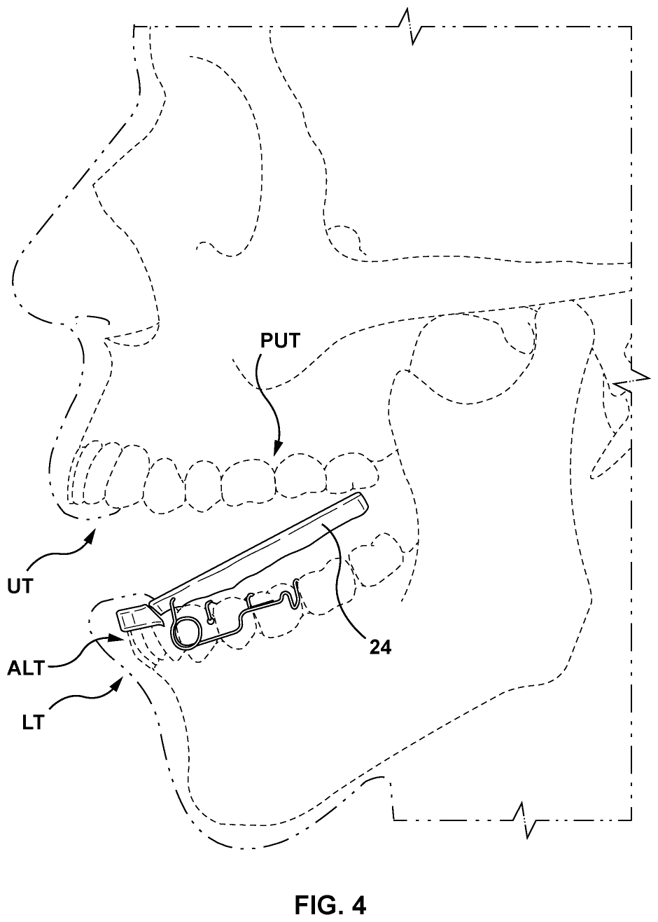

Turning now to FIGS. 1 to 5, use and operation of the dynamic dental appliance 20 for treatment of bruxism will now be described. In use, the dynamic dental appliance 20 is placed in an individual's mouth and fitted on the lower dental arch so that the retention tray 22 fits on the lower teeth LT in a snug manner with the anterior portion 32 of the retention tray 22 covering the anterior lower teeth ALT, the posterior portions 34 of the retention tray 22 covering the lingual surfaces of the posterior lower teeth PLT 34, and the retainer clasps 42 anchored on one or more lower molars. With the individual's jaw in an open position as shown in FIG. 4, the orientation of the occlusal pads 24 are set by bending the coil springs 46 and 48 of each spring mechanism 40 so that the occlusal pads 24 are angled slightly upwardly in a posterior direction to bring the distal ends of the occlusal pads 24 into contact with the back molars of the posterior upper teeth PUT. With the occlusal pads 24 set in this manner, the dynamic dental appliance 20 is in an activated state ready to absorb clenching forces and treat bruxism. Typically, in the activated state the angle of the occlusal pads 24 is set to be in the range of from about 5 degrees to about 45 degrees depending on the frequency and forcefulness of the individual's clench during bruxism.

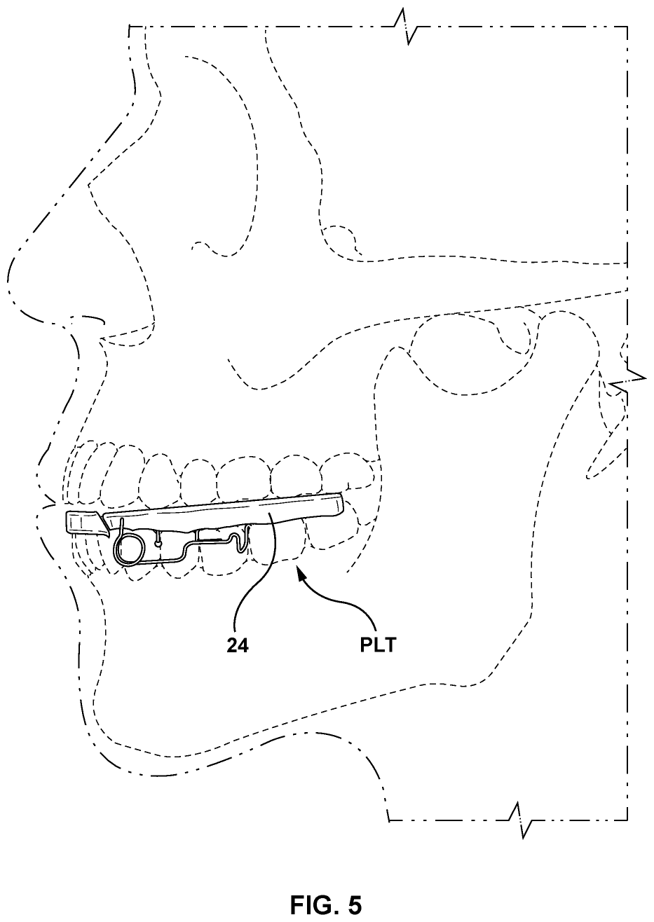

With the dynamic dental appliance 20 placed in the individual's mouth and the occlusal pads 24 set in the manner described above, when the individual clenches their jaw and brings their upper teeth UT and lower teeth LT together as shown in FIG. 5, the upwardly angled occlusal pads 24 are compressed into a flat orientation against the bias of the coil springs 46 and 48. The spring-loaded occlusal pads 24 in turn act as a shock absorber to dissipate the clenching forces before overloading the teeth and jaw muscles. Concurrently when the posterior upper molars strike the distal ends of the occlusal pads 24, the dynamic dental appliance 20 transmits a force from the occlusal pads 24 to the lower incisors. This triggers a biofeedback response from the individual's periodontal mechanoreceptors, which send a signal to the brain that interrupts the clenching. In this manner, clenching is significantly reduced or eliminated resulting in reduced wear or damage to teeth or implants, reduced jaw muscle pain, fewer sleep arousals, reduced sleep fragmentation, better overall sleep quality, and improved daytime function, cognitive function, and alertness. Over the course of bruxism treatment, as the frequency and forcefulness of the individual's clench reduce, the angle of the occlusal pads 24 can be reduced.

As mentioned above, although the dynamic dental appliance 20 has been described above as being fitted on the lower dental arch of the individual, the dynamic dental appliance 20 may alternatively be fitted on the upper dental arch of the individual. Also, if desired, dynamic dental appliances 20 may be fitted on both the upper and lower dental arches of the individual.

As will be appreciated by those of skill in the art, the extent of the force exerted between the upper and lower jaws by the occlusal pads 24 and lingual and buccal coil springs 46 and 48 to resist clenching is a function of the lingual and buccal coil spring characteristics and the upward angle of the occlusal pads 24 when set. In this embodiment, the exerted force is in the range of between about 0.1 Newton to about 400 Newtons. Smaller forces are exerted between the upper and lower jaws by the occlusal pads 24 and lingual and buccal coil springs 46 and 48 during clenching when the occlusal pads 24 are less steeply angled and closer to the flat orientation after being set and greater forces are exerted between the upper and lower jaws by the occlusal pads 24 and lingual and buccal coil springs 46 and 48 during clenching when the upward angles of the occlusal pads 24 are more steeply angled after being set.

Those of skill in the art will appreciate that the range of exertion forces can be varied by changing the spring material (i.e. using stiffer material rather than softer material or vice versa), by changing the number of coils of the lingual and buccal coil springs 46 and 48, by changing the spring configurations, and/or by changing the number of springs. For example, lingual and buccal coil springs with more than one coil may be employed and/or each spring mechanism 40 may only employ a single spring.

Still alternative biasing mechanisms are available. For example, rather than employing coil springs on the buccal and lingual sides of the dynamic dental appliance 20, each spring mechanism 40 may employ a spring-loaded adjustment device that acts between the occlusal pad 24 and the retention tray 22. The spring-loaded adjustment device may comprise a tube that houses a spring formed of stainless steel, NiTi, plastic, or other suitable material. A screw is received by the tube and engages the spring. Rotation of the screw with respect to the tube adjusts the tension of the spring within the tube allowing the force exerted between the upper and lower jaws by the occlusal pads 24 during clenching to be adjusted.

Those of skill in the art will appreciate that non-spring based biasing methods may also be employed that create a spring-like action using different materials or configuration of materials. For example, wedges of different materials, wedges of material of different densities, or combinations thereof, may be employed and inserted between the occlusal pads 24 and the retention tray 22 to provide the bias.

Alternatively, hinge mechanisms supported by folding metal or plastic wires or bars with unique properties, such as shaped memory properties, that allow the material to bend or flex between the posterior teeth and the occlusal pads, or telescopic pistons acting between the retention tray 22 and the occlusal pads 24 may be employed to provide the bias.

Although the base has been described as a retention tray that covers the lingual surface of the lower dental arch and covers the incisal surface of the lower anterior teeth, the base may on take other forms. For example, the base may be in the form of a retention mechanism with a tray or clasps made from metal or plastic or a combination of both, that anchors the dynamic dental appliance on the upper or lower dental arch and prevents it from easily dislodging off the teeth.

Although embodiments have been described above with reference to the accompanying drawings, those of skill in the art will appreciate that other variations modifications may be made.

What is claimed is:

1. A dynamic dental appliance for bruxism treatment comprising:
a base configured to fit on either the upper dental arch or the lower dental arch of an individual;
a pair of occlusal pads, one of the occlusal pads configured to overlie the left posterior teeth and the other of the occlusal pads configured to overlie the right posterior teeth; and
a biasing mechanism acting between the base and the occlusal pads, the biasing mechanism configured to exert an adjustable force against the occlusal pads when the occlusal pads are moved from a flat orientation towards a generally angled activated position.

2. The dynamic dental appliance according to claim 1, wherein the biasing mechanism comprises a pair of adjustable spring mechanisms, each adjustable spring mechanism acting between the base and a respective one of the occlusal pads.

3. The dynamic dental appliance according to claim 2, wherein each adjustable spring mechanism comprises at least one spring acting between the base and the respective one of the occlusal pads.

4. The dynamic dental appliance according to claim 3, wherein each spring is a coil spring.

5. The dynamic dental appliance according to claim 3, wherein each adjustable spring mechanism comprises a pair of springs acting between the base and the respective one of the occlusal pads, the springs being positioned on respective buccal and lingual sides of the dynamic dental appliance.

6. The dynamic dental appliance according to claim 5, wherein each spring is a coil spring.

7. The dynamic dental appliance according to claim 2, wherein the fulcrum of the adjustable spring mechanisms is located at the anterior end of each occlusal pad.

8. The dynamic dental appliance according to claim 7, wherein, in the activated position, the angle of the occlusal pads is in the range of from about 5 degrees to about 45 degrees.

9. The dynamic dental appliance according to claim 1, wherein the base is configured to fit passively on either the upper dental arch or the lower dental arch.

10. The dynamic dental appliance according to claim 9, wherein the base is in the form of a tray comprising an anterior portion configured to overlie the anterior teeth and posterior portions configured to run along the lingual sides of posterior teeth, the buccal sides of posterior teeth or both the lingual and buccal sides of the posterior teeth.

11. The dynamic dental appliance according to claim 1, wherein the occlusal pads are flat and smooth to allow the individual's teeth to skate freely thereacross.

12. The dynamic dental appliance according to claim 1, wherein the exerted adjustable force is in the range of from about 0.1 Newton to about 400 Newtons.

13. A bruxism treatment method comprising using at least one dynamic dental appliance according to claim 1 installed in an individual's mouth and fitted on either the upper dental arch, the lower dental arch, or both the upper and lower dental arches to exert an adjustable force between the individual's jaws during clenching to resist the clenching and counter brain-drive clench stimulus.

14. A bruxism treatment method comprising using at least one dynamic dental appliance fitted on either the upper dental arch, the lower dental arch or both the upper and lower dental arches of an individual that exerts an adjustable force between the individual's jaws during clenching to resist the clenching such that the at least one dynamic dental appliance absorbs and dissipates the clenching force.

15. A bruxism treatment method comprising using at least one dynamic dental appliance fitted on a dental arch of an individual that exerts an adjustable force between the individual's jaws during clenching to resist the clenching and counter brain-drive clench stimulus during sleep.

16. The bruxism treatment method according to claim 13, wherein the exerted adjustable force is in the range of from about 0.1 Newton to about 400 Newtons.

17. The bruxism treatment method according to claim 14, wherein the exerted adjustable force is in the range of from about 0.1 Newton to about 400 Newtons.

18. The bruxism treatment method according to claim 15, wherein the exerted adjustable force is in the range of from about 0.1 Newton to about 400 Newtons.

19. The bruxism treatment method according to claim 15, comprising fitting the dynamic dental appliance on either (i) the lower dental arch, (ii) the upper dental arch, or (iii) both the upper and lower dental arches.

20. The bruxism treatment method according to claim 18, comprising fitting the dynamic dental appliance on either (i) the lower dental arch, (ii) the upper dental arch, or (iii) both the upper and lower dental arches.
